(19) United States
(12) Patent Application Publication
(10) Pub. No.: US 2008/0058431 A1
(43) Pub. Date: Mar. 6, 2008

(54) Modulators of Shp2 Tyrosine Phosphatase and Their Use in the Treatment of Body Weight Disorders

(76) Inventors: Gen-Sheng Feng, San Diego, CA (US); Erquan Eric Zhang, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/594,170

(22) PCT Filed: Mar. 25, 2005
(86) PCT No.: PCT/US05/10380

§ 371 Date: Jul. 20, 2007

Related U.S. Application Data
(60) Provisional application No. 60/556,564, filed on Mar. 26, 2004.

(51) Int. Cl. G01N 33/00; A01K 67/027; A61K 45/00; A61P 3/04; C12Q 1/02

(52) U.S. Cl. 514/789; 435/29; 800/18; 800/3

(57) ABSTRACT

Described are methods and compositions for regulating body weight and/or regulating the rate of weight gain or loss, and particularly, for treating or preventing obesity. Specifically, methods of administering varying levels of Shp2 modulators to an animal, alone or in combination with body weight regulating agents are disclosed. Methods and compositions for treating a variety of disorders associated with or caused by undesirable body weight are also described. Also described are methods for identifying compounds useful for regulation of body weight and associated conditions. In particular, methods are disclosed for identification of compounds that preferentially modulate binding of Shp2 to leptin receptors. Also described is a genetically modified non-human animal model for studying the peripheral and central pathways of energy homeostasis. Also disclosed are methods of identifying compounds for regulating such pathways.

### GOVERNMENTAL INTEREST

This invention was made with United States Government support under grant number GM053660 awarded by the National Institutes of Health. The U.S. Government has certain rights in this invention.

### FIELD OF THE INVENTION

This invention relates to modulation of Shp2 tyrosine phosphatase activity to treat body weight disorders.

### BACKGROUND OF THE INVENTION

The regulation of body weight, and particularly, obesity and conditions related thereto, is a major health concern throughout the world, and particularly in the United States, contributing to morbidity and mortality.

Obesity is a metabolic disorder characterized by excessive accumulation of fat stores in adipose tissue. Obesity is associated with diseases such as diabetes, hypertension and heart disease, whose incidence increases with body-mass index (BMI, body mass in kg/square of height in meters).

Generally, obesity is due to energy intake that exceeds energy expenditure. This can be caused by overeating, i.e. higher food intake than necessary for maintenance of body mass. In addition, low mobility and low metabolic rate may predispose for obesity (see Flier, J. S. and Foster D. W. 1998 Eating disorders: obesity anorexia nervosa and bulimia nervosa, Williams Textbook of Endocrinology, 9th Ed, Saunders Co.).

However, the general opinion that obesity is largely the result of a lack of willpower is unsatisfactory. For this reason, intense research efforts have been made to reveal the genetic and environmental factors of importance for development of obesity (Friedman J. M. & Halaas J. L. 1998 Nature 395:763-70).

Animal models can be used for investigation of which genes are related to the development of obesity. Of particular importance is the information that can be gained from mouse strains that develop obesity because of gene knockouts. These mouse strains can provide evidence that a certain gene product is of crucial importance for regulating body fat. This in turn may facilitate the development of new treatment paradigms. There are indications that there are gender differences regarding the genetic ethiology of obesity (see e.g., Costet, P. et al. 1998 J Biol Chem 273:29577-29585).

Following the cloning of leptin in 1994 (see, Zhang et al. 1994 Nature 372:425-432), there were great hopes that this would mean new possibilities to treat obesity and overeating. However, later it was found that obesity in humans very seldom is due to leptin deficiency, but rather is associated with increased leptin levels. Moreover, it has been shown that both mice and humans often are resistant to the antiobesity effect of leptin (see, e.g., Flier, J. S. 1998 J Clin Endocr Metab 83:1407-1413).

The 16 kDa protein leptin is predominantly produced in white adipocytes from which leptin is then released into circulation. Leptin production by fat cells and circulating plasma leptin levels are highly correlated with adipose tissue mass (Flier J. S. 1997 PNAS USA 94:4242-5). Leptin acts through specific receptors in the hypothalamus to create a feedback loop for body weight regulation. Therefore, the pathophysiology of obesity was assumed to be partly endocrine. However, leptin levels do not rise significantly after a meal, and also do not result in the termination of a meal. Instead leptin appears largely to exert long-term effects on food consumption and energy expenditure (Flier, J. S. 1998 J Clin Endocr Metab 83:1407-1413; Friedman J. M. & Halaas J. L. 1998 Nature 395:763-70).

Obese (ob) mice which lack leptin show many of the abnormalities seen in starved animals, including hyperphagia, decreased body temperature, decreased energy expenditure, decreased immune function, and infertility.

Leptin replacement corrects all of these abnormalities implying that ob mice live in a state of “perceived starvation” due to lack of leptin and that the biological response in the presence of food leads to obesity. These observations led to speculation that leptin's main physiological role is to signal nutritional status during periods of food deprivation (Flier, J. S. 1998 J Clin Endocr Metab 83:1407-1413; Friedman J. M. & Halaas J. L. 1998 Nature 395:763-70).

The leptin receptor (Ob-R) is normally expressed at high levels in hypothalamic neurons and in other cell types, including T cells and vascular endothelial cells. In situ hybridization was used to identify the hypothalamic arcuate nucleus, and also dorsomedial hypothalamic nucleus (DMH), paraventricular nucleus (PVN), ventromedial hypothalamic nucleus (VMH) and lateral hypothalamic nucleus (LH) as principal sites of Ob-R expression in the central nervous system. Each of these nuclei, such as the arcuate nucleus, express one or more neuropeptides and neurotransmitters such as neuropeptide Y (NPY) and melanocyte-stimulating hormone alpha (α-MSH), that regulate food intake and/or body weight, probably by actions downstream of leptin (Friedman J. M. & Halaas J. L. 1998 Nature 395:763-70; Flier J. S. & Maratos-Flier E. 1998 Cell 92:437-40).

The role of leptin in the pathogenesis of obesity may be inferred by measuring plasma leptin levels. An increase in plasma leptin suggests that obesity is the result of resistance to leptin. A low or normal plasma concentration of leptin suggests that obesity is due to decreased production of leptin. This interpretation is similar to that used in studies of insulin and the pathogenesis of type I and type II diabetes. As is the case with insulin and its receptor in diabetes, mutations of leptin and its receptor are rare in human obesity, but most obese individuals still have higher levels of leptin than do non-obese individuals, an indication of leptin resistance that might be receptor-independent (Flier J. S. 1997 PNAS USA 94:4242-5).

Leptin activates the leptin receptor long form (ObRb) in the hypothalamus for control of food intake, metabolism and neuroendocrine response to nutritional alteration (Y. Zhang et al. 1994 Nature 372:425-32; J. M. Friedman, & J. L. Halaas 1998 Nature 395:763-70). This hormone regulates mammalian food consumption by activating the expression of anorexic gene products, such as proopiomelanocortin (POMC), and repressing the expression of orexigenic peptides neuropeptide Y (NPY) and agouti-related protein (AgRP) (Cowley M. A. et al. 2001 Nature 411:480-4; M. W. Schwartz et al. 2000 Nature 404:661-71). However, little is known about the role of leptin in controlling metabolism, which is distinct from its anorectic effect (S. Kamohara et al. 1997 Nature 389:374-7; N. Levin et al. 1996 PNAS USA 93:1726-30). A recent report suggests a mechanism for the leptin's metabolic action by down-regulation of stearoyl-CoA desaturase-1 (SCD-1)in the liver (P. Cohen et al. 2002 Science 297:240-3).

Many genes involved in development of obesity have recently been found and most of them seem to act downstream of leptin at the hypothalamic level. Other genes that are involved in development of obesity encode neuropeptides, e.g., leukocyte adhesion receptors, which are important cell-cell adhesion molecules in the inflammatory and immune systems (Dong Z. M. et al. 1997 PNAS USA 94:7526-30), and neurocytokines like ciliary neurotrophic factor, whose receptor subunits share sequence similarity with the leptin receptor (Gloaguen I. et al. 1997 PNAS USA 94:6456-61). The identification of anti-obesity mechanisms that act independently or together with the leptin system may help to develop strategies for the treatment of obesity associated with leptin resistance.

In deciphering the proximal signals of ObRb, the Sh2-containing tyrosine phosphatase Shp2 has been shown to bind the ligand-activated receptor through phosphorylated Tyr985 (C. Li, & J. M. Friedman 1999 PNAS USA 96:9677-82; L. R. Carpenter et al. 1998 PNAS USA 95:6061-6), while Tyr1138 serves as a docking site for the transcription factor Stat3 (N. Ghilardi et al. 1996 PNAS USA 93:6231-5; H. Baumann et al., 1996 PNAS USA 93:8374-8). Injection of leptin into mice induced Stat3 activation specifically in the hypothalamus (C. Vaisse et al. 1996 Nat Genet 14:95-7, and disrupted the ObRb/Stat3 interaction by replacing Tyr1138 with a Ser residue caused hyperphagia and obesity in mutant mice, indicating a requirement of Stat3 for leptin regulation of food intake and energy homeostasis (S. H. Bates et al. 2003 Nature 421:856-9). In vitro biochemical analysis also suggested a negative effect of Shp2 in modulating leptin-induced Jak2 or Stat3 signals (C. Li, & J. M. Friedman 1999 PNAS USA 96:9677-82; L. R. Carpenter et al. 1998 PNAS USA 95:6061-6).

On the other side of the spectrum of body weight problems, other individuals suffer from one or more “wasting” disorders (e.g., wasting syndrome, cachexia, sarcopenia) which cause undesirable and/or unhealthy loss of weight or loss of body cell mass. In the elderly, as well as in AIDS and cancer patients, wasting disorders can result in undesired loss of body weight, including both the fat and the fat-free compartments.

Body weight disorders, such as anorexia nervosa and bulimia nervosa which together affect approximately 0.2% of the female population of the western world, also pose serious health threats. Wasting diseases can be the result of inadequate intake of food and/or metabolic changes related to illness and/or the aging process. Cancer patients and AIDS patients, as well as patients following extensive surgery or having chronic infections, immunologic diseases, hyperthyroidism, extraintestinal Crohn's disease, psychogenic disease, chronic heart failure or other severe trauma, frequently suffer from wasting disease which is sometimes also referred to as cachexia, a metabolic and, sometimes, an eating disorder. Cachexia is additionally characterized by hypermetabolism and hypercatabolism.

Cachexia, a potentially lethal syndrome afflicting marnmals, frequently complicates the treatment of infection, inflammation and cancer. It is characterized by profound weight loss caused by wasting of body fat (adipose) and muscle (protein) (Tracey et al. 1988 J Exp Med 167:1211-1227; Lawson et al. 1982 Ann Rev Nutr 2:277-301). Anorexia, anemia, and weakness may also occur in cachexia (Tracey et al., supra). Cachexia may further be characterized by, inter alia, depression of glucose level (hypoglycemia) and elevation of triglyceride level (hypertriglyceridemia). Moreover, the syndrome is not alleviated by adequate caloric uptake. Indeed, weight loss may continue in cachexia even while an adequate diet is consumed (Silva et al. 1988 J General Microbiology 134:1629-1633).

It is an objective of the invention to provide modulators of body weight, to provide therapy for body weight disorders, and to provide assay systems for the screening of substances that can be used to control body weight.

### SUMMARY OF THE INVENTION

Embodiments of the invention relate to improved therapies and methods for reducing or preventing body weight disorders in a mammal. In particular, methods for identifying or selecting compounds that modulate Shp2 activity and thus are useful for controlling the total body weight and percentage of body fat in a mammal (e.g., a human) are disclosed.

Accordingly, one aspect of the invention includes a screening method for determining whether a compound is useful for treating, stabilizing, or preventing a higher than desired total body weight or a higher than desired percentage of body fat in a mammal. This method involves measuring Shp2 activity in a cell, tissue, or mammal in the presence and absence of the compound. The compound is determined to treat, stabilize, or prevent a higher than desired total body weight or a higher than desired percentage of body fat if the compound increases Shp2 activity or binds to a Shp2 binding site on the leptin receptor. In some embodiments, the method also includes administering the compound to a mammal in need of the treatment (e.g., an obese mammal or a mammal with excess fat). In certain embodiments, the compound is a member of a library of at least 5, 10, 15, 20, 30, 50, or more compounds, all of which are simultaneously administered to the cell, tissue, or mammal. Preferably, the compound increases the level of Shp2 MRNA or protein, an activity of Shp2, the half-life of Shp2 mRNA or protein, or the binding of Shp2 to a leptin receptor. In a preferred embodiment, the compound is a Shp2 agonist. Preferably, the level of Shp2 MRNA or protein, an activity of Shp2, the half-life of Shp2 mRNA or protein, or the binding of Shp2 to a leptin receptor) increases by at least 5, 10, 20, 30, 40, 50, 60, or 80%.

In another aspect, the invention features improved methods for reducing or preventing undesired, excess body fat in a mammal. In particular, these methods involve administering a compound that increases Shp2 activity to the mammal.

Embodiments of the invention provide a number of advantages related to reducing or stabilizing the amount of body fat in a mammal. These methods are desirable because they may be used to obtain a significant, long-term reduction in body fat. The therapies described herein are expected to have few, if any, adverse side effects.

Another embodiment of the invention is a method for treating obesity, leptin resistance and dyslipidemia in a mammal, including a human, by administering to the mammal in need of such treatment a therapeutically effective amount of any combination of two or more of the following compounds: a compound or combination of compounds that activates Shp2, an anti-diabetic compound, and a lipid-lowering agent.

Another embodiment of the invention is a method for increasing body fat in a mammal in need thereof. In particular, these methods involve administering a compound that decreases Shp2 activity to the mammal.

Accordingly, in another aspect, the invention includes a screening method for determining whether a compound is useful for treating, stabilizing, or preventing a lower than desired total body weight or a lower than desired percentage of body fat in a mammal. This method involves measuring Shp2 activity in a cell, tissue, or mamrnal in the presence and absence of the compound. The compound is determined to treat, stabilize, or prevent a lower than desired total body weight or a lower than desired percentage of body fat if the compound decreases Shp2 activity or competes with Shp2 for the binding site on the leptin receptor. In some embodiments, the method also includes administering the compound to a mammal in need of the treatment (e.g., an anorexic or cachexic mammal). In certain embodiments, the compound is a member of a library of at least 5, 10, 15, 20, 30, 50, or more compounds, all of which are simultaneously administered to the cell, tissue, or mammal. Preferably, the compound decreases the level of Shp2 mRNA or protein, an activity of Shp2, the half-life of Shp2 mRNA or protein, or the binding of Shp2 to a leptin receptor. In a preferred embodiment, the compound is a Shp2 antagonist. Preferably, the level of Shp2 MRNA or protein, an activity of Shp2, the half-life of Shp2 mRNA or protein, or the binding of Shp2 to a leptin receptor) decreases by at least 5, 10, 20, 30, 40, 50, 60, or 80%.

Another object of the invention is a pharmaceutical composition for the treatment of body weight disorders, e.g., obesity-related disorders and disorders associated with excessive weight loss comprising: a pharmaceutically acceptable carrier and a therapeutically effective amount of a compound or combination of compounds that modulate Shp2 activity.

The obesity-related diseases or disorders envisioned to be treated by the methods of the invention include, but are not limited to, hyperlipidemia, atherosclerosis, diabetes, and hypertension. The disorders associated with excessive weight loss and envisioned to be treated by the methods of the invention include, but are not limited to, cachexia, cancer-related weight loss, AIDS-related weight loss, chronic inflammatory disease-related weight loss, and anorexia.

Other features and advantages of the invention will be apparent from the following detailed description and from the claims.

### BRIEF DESCRIPTION OF THE DRAWINGS

FIG. 1. CaSKO mice are obese. (A, B) Body weights (BW) of Shp2 knockout (CaSKO) and control mice, measured at the indicated time points. Data are expressed as the means with SEM of at least 12 mice of each gender and genotype. Starting from P32 in males and P28 in females, there were significant differences between the CaSKO mice and controls/heterozygotes ( [symbol] , P<0.01 in an unpaired Student's t test). Wild-type (+/+, F/F; diamond); heterozygous mice (Cre, F/+; square); CaSKO (Cre, F/F; triangle). (C) White and brown adipose tissue (WAT and BAT) mass was assessed in mice at age of 8 weeks. Data represent the mean±SEM of at least 8 mice of each gender and genotype (**, P<0.01). (D) leptin concentrations, determined in mouse serum samples collected at age of 8 weeks by enzyme-linked immunosorbent assay (ELISA). Data represent the mean +SEM of at least 8 mice of each gender and genotype (**, P<0.01).

FIG. 2. Leptin-induced signals are directly interfered in CaSKO mice. No significant changes in the mRNA levels of proopiomelanocortin (POMC) were observed between controls and CaSKO mice. The expression of orexigenic peptide neuropeptide Y (NPY) mRNA level was increased 2-3 folds in control mice after 20 hr fasting, with no increase detected in CaSKO mice. **: P<0.01, *: P<0.05.

FIG. 3. The obesity is caused primarily by altered metabolism. (A) Gain in body weight and total food intake in wild type and CaSKO mice (**, P<0.0002 with at least 8 mice per group). (B, C) Blood glucose and serum insulin concentrations measured at age of 8 weeks, in the status of either fed or fasted for 20 hr (*, P<0.05; **, P<0.01 with at least 8 mice each group). (D) Relative liver weight versus body weight (mean +SEM) of at least 8 mice for each gender and genotype (**, P=0.002).

FIG. 4. Dysfunction of the hypothalamus-pituitary axis in CaSKO mice. (A) Adrenal hormone corticosterone, measured by enzyme immunoassay (EIA); (B) thyroid stimulating hormone, TSH, and (C) growth hormone, GH, determined by radioimmunoassay (RIA). **: P<0.01, *: P<0.05.

FIG. 5. Generation of conditional Shp2 knockout mice. The gene targeting strategy is shown.

FIG. 6. Normal sizes of control organs. Mass of heart (A), spleen (B), and kidney (C) is shown. Data represent the mean±SEM of at least 8 mice of each gender and genotype, and P value is greater than 0.2 in each analysis.

FIG. 7. Normal BDNF expression in hypothalamus of CaSKO mice.

### DETAILED DESCRIPTION OF THE PREFERRED EMBODIMENT

One embodiment of the invention provides a method of treating, stabilizing, or preventing a higher than desired total body weight or a higher than desired percentage of body fat in a mammal (e.g., a human) that involves administering to the mammal a compound that increases Shp2 activity in an amount sufficient to treat, reduce, or prevent a higher than desired total body weight or a higher than desired percentage of body fat. Preferably, the compound increases the level of Shp2 mRNA or protein, an activity of Shp2, the half-life of Shp2 mRNA or protein, or the binding of Shp2 to a leptin receptor. In a preferred embodiment, the compound is a Shp2 agonist. In a preferred embodiment, Shp2 activity is increased in neurons. In another preferred embodiment Shp2 activity is increased in the forebrain. In yet another preferred embodiment Shp2 activity is increased in the hypothalamus.

In another aspect, the invention provides a method of treating, stabilizing or preventing a lower than desired total body weight or a lower than desired percentage of body fat in a mammal that involves administering to the mammal a compound that decreases Shp2 activity in an amount sufficient to treat or prevent lower than desired total body weight or lower than desired percentage of body fat. Preferably, the compound decreases the level of Shp2 mRNA or protein, an activity of Shp2, the half-life of Shp2 mRNA or protein, or the binding of Shp2 to a leptin receptor. In a preferred embodiment, the compound is a Shp2 antagonist. In a preferred embodiment, Shp2 activity is decreased in neurons. In another preferred embodiment Shp2 activity is decreased in the forebrain. In yet another preferred embodiment Shp2 activity is decreased in the hypothalamus.

Embodiments of the invention relate to the discovery that specific ablation of the Shp2 gene in forebrain neurons caused resistance to leptin in mice. This leptin resistance was characterized by early-onset obesity and increased serum levels of leptin, insulin arid triglycerides. The mutant animals, however, did not show hyperphagia and were hyperglycemic in the fed state while hypoglycemic when fasted. Furthermore, the male mutant mice developed hepatomegaly, with increased lipid content, up-regulated anabolic gene expression and impaired catabolic gene expression in the liver. Basal and leptin-induced Stat3 activation in the hypothalamus was enhanced, while leptin-stimulated signals from phosphorylated extracellular signal-regulated kinases (phospho-Erk) in arcuate nucleus were reduced in the absence of Shp2. Thus, it appears that the primary function of Shp2 in the hypothalamus is to promote the metabolic activity of leptin in energy balance through activation of kinases such as Erk.

In some embodiments, at least 2, 3, 4, 5, or more compounds that modulate Shp2 activity are administered to the mammal. Preferably, the one or more compounds are administered intravenously, parenterally, subcutaneously, intramuscularly, ophthalmicly, intraventricularly, intraperitoneally, orally, topically, or intranasally to the mammal. In a preferred embodiment a compound that modulate Shp2 activity is conjugated to a molecule that promotes penetration of the compound through a Blood-Brain Barrier. In preferred embodiments, the one or more compounds are administered using an extended release device. In other preferred embodiments, an additional compound is administered to the mammal that inhibits angiogenesis, and adipogenesis, or alters appetite.

In one embodiment, the mammal treated with the methods of the invention is obese. Preferably, the percentage of body fat in the mammal treated with a Shp2 activator decreases by at least 5, 10, 20, 30, 40, 50, 60, or 80%. In other preferred embodiments, total body weight of the mammal decreases by at least 5, 10, 20, 30, 40, 50, or 60%. Preferably, the number of cells other than adipocytes or endothelial cells decreases by less than 50, 40, 30, 20, 10 or 5%. In other preferred embodiments, the compound does not effect the viability or proliferation of cells other than adipocytes or endothelial cells.

In another embodiment, the mammal treated with the methods of the invention suffers from low body weight. Preferably, the percentage of body fat in a mammal treated with a Shp2 inhibitor increases by at least 5, 10, 20, 30, 40, 50, 60, or 80%. In other preferred embodiments, total body weight of the mammal increases by at least 5, 10, 20, 30, 40, 50, 60 or 80%. Preferably, the number of cells other than adipocytes or endothelial cells increases by at least 5, 10, 20, 30, 40 or 60%.

With respect to the therapeutic methods of the invention, the administration of one or more compounds to a mammal is not limited to a particular mode of administration, dosage, or frequency of dosing. All modes of administration are contemplated, including intramuscular, intravenous, intraarticular, intralesional, subcutaneous, or any other route sufficient to provide a dose adequate to prevent or treat a higher than desired total body weight or a higher than desired percentage of body fat.

Preferably the modulators of shp2 activity increase the activity of the neuronal Shp2.

Preferably, the modulators of Shp2 activity are capable of traversing the Blood-Brain Barrier.

Preferably, the modulators of Shp2 activity are conjugated with a Blood-Brain Barrier delivery targeting vector such as, for example, avidin-biotin linked chimeric peptide, monoclonal antibody to the transferrin receptor, transferrin, L-Glutamate; short natural-derived peptides that are able to cross efficiently the BBB without compromising its integrity; antibody-avidin fusion protein, etc. (Song B. et al. 2002 J Pharmacol Exp Ther 301:605-10; Pardridge W. M. et al. 2001 Jpn J Pharmacol 87:97-103 and references therein; Liao G. S. et al. 2001 J Nat Toxins 10:291-7; Sakaeda T. et al. 2001 J Drug Target 9:23-37; Rousselle C. et al. 2001 J Pharmacol Exp Ther 296:124-31; Penichet M. L. et al. 1999 J Immunol 163:4421-6; or lipid nanoparticles as described, for example in Olbrich C. et al. 2002 J Drug Target 10:387-96.

These methods may be used to treat humans or any domesticated or farm animal. Examples of preferred mammals include humans, cows, sheep, big-horn sheep, goats, buffaloes, antelopes, oxen, horses, donkeys, mule, deer, elk, caribou, water buffalo, camels, llama, alpaca, rabbits, pigs, mice, rats, guinea pigs, hamsters, dogs, cats, and primates. The compound(s) may be administered to the mammal in a single dose or multiple doses. When multiple doses are administered, the doses may be separated from one another by, for example, one day, one week, one month, or one year. It is to be understood that, for any particular subject, specific dosage regimes should be adjusted over time according to the individual need and the professional judgment of the person administering or supervising the administration of the compositions. If desired, conventional treatments such as appetite suppressants or stimulants, diet, exercise as well as anti-depressants may be used in combination with the compounds of the present invention.

As used herein by “treating, stabilizing, or preventing a higher than desired total body weight or a higher than desired percentage of body fat” is meant preventing or delaying an initial or subsequent occurrence of a higher than desired weight or percentage of body fat, or stabilizing or reducing a subject's total body weight or percentage of body fat. Obesity is typically classified as mild (i.e., 20 to 40% overweight based on the rmidpoint of the weight range for the subject's height on a standard height-weight table), moderate (i.e., 41 to 100% overweight), or severe (i.e., over 100% overweight).

In some embodiments, the subject's body mass index (i.e., weight in kilograms divided by height in meters squared) is greater than 20, 25, 30, 35, 40, or 45 kg/m². In certain embodiments, the subject has an increased body weight or an increased percentage of body fat due to, at least in part, a hormonal or metabolic disorder (e.g., a thyroid disorder), brain damage (e.g., damage to the hypothalamus), an adverse side-effect from a medication, or a genetic factor.

In some embodiments, the subject has a binge eating disorder, bulimia nervosa, or anorexia nervosa.

Desirably, administration of a compound to the subject results in a decrease of at least 5, 10, 20, 30, 40, 50, or 60% in the subject's total body weight or weight due to body fat. Preferably, the decrease in muscle mass is less than 50, 40, 30, 20, 10, 5, or 3%. In other preferred embodiments, the decrease in body fat or total body weight leads to a decrease in blood pressure, incidence or severity of diabetes, or incidence or severity of coronary artery disease (e.g., heart attacks).

By “compound that modulates Shp2 activity” is meant a compound that increases or decreases the level of Shp2 mRNA or protein, an activity of Shp2 (e.g., phosphatase activity), the half-life of Shp2 mRNA or protein, or the binding of Shp2 to a leptin receptor, as measured using standard methods (see, for example, Ausubel et al., Current Protocols in Molecular Biology, Chapter 9, John Wiley & Sons, New York, 2000). Shp2 A expression levels may be determined using standard RNase protection assays or in situ hybridization assays, and the level of Shp2 protein may be determined using standard Western or immunohistochemistry analysis with an anti-Shp2 antibody (see, for example, Ausubel et al., supra). In other preferred embodiments, a compound that increases Shp2 activity increases or stabilizes the level of mRNA or protein, or the phosphorylation level of a signal transduction protein. The level of Shp2 activity may be determined by measuring the change in total body weight or percentage of body fat using standard assays, such as those described herein. Compounds that may be tested for their ability to modulate Shp2 activity include, but are not limited to, synthetic organic molecules, naturally occurring organic molecules, nucleic acid molecules, biosynthetic proteins or peptides, naturally occurring peptides or proteins. Preferably, the compound increases or decreases Shp2 activity by at least 20, 40, 50, 60, 80, or 90%. In another preferred embodiment, the level of Shp2 activity is at least 2, 3, 5, 10, 20, or 50-fold higher or lower in the presence of the compound.

By “increasing or decreasing expression or activity” is meant increasing or decreasing expression or activity, for example, of a protein or nucleic acid, relative to control conditions.

The modulation in expression or activity is preferably an increase of at least 20, 40, 50, 75, 90, 100, 200, 500, or even 1000%, or decrease of at least 10, 20, 40, 50, 60, 70, 80, or 90%. In various embodiments, transcription, translation, mRNA or protein stability, or the binding of the mRNA or protein to other molecules in vivo is increased or decreased by the therapy. The level of mRNA may be determined by standard Northern blot analysis, and the level of protein may be determined by standard Western blot analysis, such as the analyses described herein or those described by, for example, Ausubel et al. (Current Protocols in Molecular Biology, John Wiley & Sons, New York, 2000). In one embodiment, the level of a protein is determined by measuring the level of enzymatic activity, using standard methods. In another preferred embodiment, the level of mRNA, protein, or enzymatic activity is equal to or more than 20, 10, 5, or 2-fold above the corresponding basal level in from a control mammal with a normal percentage of body fat. In another preferred embodiment, the level of mRNA, protein, or enzymatic activity is equal to or less than 0.5, 0.4, 0.3, 0.2 or 0.1 of the corresponding basal level in from a control mammal with a normal percentage of body fat.

By “specifically binding a protein” is meant binding to the protein (e.g., Shp2 or leptin receptor), but not substantially binding to other molecules in a sample, e.g., a biological sample, that naturally includes the protein.

Other embodiments of the invention include methods for treating body weight disorders, e.g., obesity, and leptin resistance, or wasting disorder in mammals through administration of a pharmacological composition containing an agent which modulates: (1) the activity of the Shp2 protein, or (2) expression of the Shp2 gene, or (3) expression of Shp2 regulated target genes (or any combination of the above). The modulation of Shp2 may be achieved through: (1) direct binding of a pharmacological agent (a Shp2 agonist or antagonist) to the Shp2 protein and modulation of its activation potential, or (2) through modulating a productive association of Shp2 with the leptin receptor, or (3) regulating the expression of the Shp2 gene, or (4) selectively modulating its activity in a tissue through promoting the binding of a co-activator, or inhibiting the binding of a co-repressor, or any combination of the above. The resulting product of these changes may include any combination of (but are not limited to): (1) prevention of weight gain, (2) weight loss, (3) prevention of weight loss, (4) weight gain, and (5) improvement in leptin resistance.

Embodiments of the invention also include a method involving the use of a combination of a Shp2 agonist with anti-diabetic agents such as, but not limited to, metformin and/or a sulfonylurea to control insulin resistance and type 2 diabetes in obese insulin resistant/type 2 diabetes patients. Since most obese diabetic individuals also suffer from dyslipidemia and cardiovascular disease, a combination of a Shp2 agonist, an anti-diabetic agent and a lipid lowering agent such as a PPARα agonist (such as, but not limited to fenofibrate and gemfibrozil) and a HMG-CoA reductase inhibitor (such as, but not limited to, pravastatin, lovastatin, simvastatin and atorvastatin) may be used to reduce hyperlipidemia and cardiovascular diseases.

Another embodiment of the invention includes a treatment method involving the use of a combination of a Shp2 antagonist with appetite stimulants or anti-depressants to promote healthy weight gain in patients suffering from abnormal weight loss.

Other embodiments of the invention include methods for screening and identifying compounds that bind to and/or regulate Shp2.

### Screening Assays for Compounds that Modulate Shp2 Expression or Activity

The following assays identified compounds that interacted with Shp2. Also described are assays that identified compounds that interfered with the interaction of Shp2 and its natural ligands, e.g., leptin receptor, transmembrane or intracellular proteins involved in Shp2-mediated signal transduction, and to compounds which modulated the activity of the Shp2 gene (see, for example, Sui, G. et al. 2002 PNAS USA 99:5515-5520). Assays may additionally be utilized which identify compounds that bind to Shp2 gene regulatory sequences and modulate Shp2 gene expression (see, for example, Platt, K. A. 1994 J Biol Chem 269:28558-28562).

Compounds which bind to Shp2 include, but are not limited to, peptides, antibodies and fragments thereof, and other organic compounds (such as for example, peptidomimetics) that bind to Shp2 and can inhibit the activity triggered by its natural ligand (i.e., antagonists); as well as peptides, antibodies or fragments thereof, and other organic compounds that mimic the active site of the Shp2 (or a portion thereof) and bind to and “neutralize” a natural ligand.

Such compounds may include, but are not limited to, peptides such as, for example, soluble peptides, including but not limited to members of random peptide libraries (see, for example, Lam, K.S. et al. 1991 Nature 354:82-84; Houghten, R. et al. 1991 Nature 354:84-86), and combinatorial chemistry-derived molecular library made of D- and/or L-configuration amino acids, phosphopeptides and antibodies. In one embodiment, the antibodies include polyclonal, monoclonal, humanized, anti-idiotypic, chimeric or single chain antibodies. Moreover, FAb, F(ab′)₂ and FAb expression library fragments, and epitope-binding fragments thereof are also contemplated. Other embodiments include small organic or inorganic molecules which may be screened, as described herein.

Other Shp2 binding compounds include, but are not limited to, small organic molecules and polynucleotides that are able to gain entry into an appropriate cell and affect the expression of the Shp2 gene or some other gene involved in the Shp2 signal transduction pathway. Compounds that affect the activity of Shp2 by inhibiting the enzymatic activity of Shp2 or the activity of other intracellular factors involved in the Shp2 signal transduction pathway are also within the scope of the invention. Compounds that affect the activity of Shp2 by enhancing the enzymatic activity of Shp2 or the activity of some other intracellular factor involved in the Shp2 signal transduction pathway are also within the scope of the invention.

Computer modeling and searching technologies permit identification of compounds, or the improvement of already identified compounds, that can modulate Shp2 expression or activity. Having identified such a compound or composition, the active sites or regions can be identified. Such active sites might typically be ligand-binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the Shp2 polypeptide the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds of Shp2 can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential Shp2 modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of Shp2 natural ligands, Shp2, and related transduction and transcription factors will be apparent to those of skill in the art.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al. 1988 Acta Pharmaceutical Fennica 97:159-166; Ripka, 1988 New Scientist 54-57; McKinaly and Rossmann 1989 Annu Rev Pharmacol Toxicol 29:111-122; Perry and Davies 1989 OSAR: Quantitative Structure-Activity Relationships in Drug Design pp. 189-193 Alan R. Liss, Inc.; Lewis and Dean 1989 Proc R Soc Lond 236:125-140 and 141-162; and, with respect to a model receptor for nucleic acid components, Askew, et al. 1989 J Am Clein Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators, preferably inhibitors.

Compounds identified via assays such as those described herein may be useful, for example, in modulating Shp2 interaction with the leptin receptor.

### In Vitro Cell-Free Screening Assays for Compounds that Bind to Shp2.

In vitro systems may be used to identify compounds capable of interacting with Shp2. These compounds may be useful, for example, in modulating the activity of wild-type and/or mutant Shp2 gene products. In addition, these compounds may be useful in screens for identifying compounds that disrupt normal Shp2 interactions, e.g., with leptin receptor. Alternatively, the compounds themselves may disrupt such interactions.

The assays used to identify compounds that bind to Shp2 involve preparing a reaction mixture of Shp2 and the test compound under conditions and for a time sufficient to allow the two components to interact, thus forming a complex which can be removed and/or detected in the reaction mixture. The Shp2 species used can vary depending upon the goal of the screening assay. For example, where antagonists of the natural ligand are sought, the full length Shp2, or a peptide corresponding to the Shp2 active site, or a fusion protein containing the Shp2 active site fused to a protein or polypeptide that affords advantages in the assay system can be utilized. Such assay system may be, but not limited to labeling, isolation of the resulting complex, etc.

The screening assays can be conducted in a variety of ways. For example, one method to conduct such an assay would involve anchoring the Shp2 protein, polypeptide, peptide or fusion protein or the test substance onto a solid phase and detecting Shp2/test compound complexes anchored on the solid phase at the end of the reaction. In one embodiment of such a method, the Shp2 reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface. In one embodiment, a labeled antibody specific for the previously nonimmobilized component is used. The antibody, in turn, may be directly labeled or indirectly labeled with a labeled anti-Ig antibody.

Alternatively, a reaction can be conducted in a liquid phase, the reaction products separated from unreacted components, and complexes detected. In one embodiment, an immobilized antibody specific for the Shp2 protein, polypeptide, peptide or fusion protein or the test compound is used to anchor any complexes formed in solution, and a labeled antibody specific for the other component of the possible complex is used to detect anchored complexes.

Alternatively, cell-based assays, membrane vesicle-based assays and membrane fraction-based assays can be used to identify compounds that interact with Shp2. To this end, cell lines that express Shp2, or cell lines that have been genetically engineered to express Shp2 can be used.

### Assays for Intracellular Proteins that Interact with the Shp2.

Any method suitable for detecting protein-protein interactions may be employed for identifying transmembrane proteins or intracellular proteins that interact with Shp2. Among the traditional methods which may be employed are co-immunoprecipitation, crosslinking and co-purification through gradients or chromatographic columns of cell lysates or proteins obtained from cell lysates and the Shp2 to identify proteins in the lysate that interact with the Shp2. For these assays, the Shp2 component used can be a full-length Shp2, a peptide corresponding to the active site of Shp2, or a fusion protein containing the active site of Shp2.

Once isolated, such an intracellular protein can be identified and can, in turn, be used, in conjunction with standard techniques, to identify proteins with which it interacts. For example, at least a portion of the amino acid sequence of an intracellular protein which interacts with the Shp2 can be ascertained using techniques well known to those of skill in the art, such as via the Edman degradation technique (see, for example, Creighton, 1983 Proteins: Structures and Molecular Principles, W. H. Freeman & Co. N.Y. pp. 34-49). The amino acid sequence obtained may be used as a guide for generating oligonucleotide mixtures that can be used to screen for gene sequences encoding such intracellular proteins. Screening may be accomplished, for example, by standard hybridization or well-known PCR techniques. Techniques for the generation of oligonucleotide mixtures and the screening are well-known (see, for example, Ausubel, F. M. et al. eds. 1989 Current Protocols in Molecular Biology Green Publishing Associates Inc., and John Wiley & sons, Inc. New York; and Innis, M. et al., eds. 1990 PCR Protocols: A Guide to Methods and Applications, Academic Press, Inc., New York)

Additionally, methods may be employed which result in the simultaneous identification of genes which encode the transmembrane or intracellular proteins interacting with Shp2. These methods include, for example, probing expression libraries, in a manner similar to the well-known technique of antibody probing of λgt11 libraries, using labeled Shp2 protein, or a Shp2 polypeptide, peptide or fusion protein. Such fusion protein may be a Shp2 polypeptide or Shp2 domain fused to a marker such as an enzyme, fluor, luminescent protein, or dye. Alternatively, such fusion protein may be a Shp2 polypeptide or Shp2 domain fused to an Ig-Fc domain.

One method which detects protein interactions in vivo, the two-hybrid system, is described in detail for illustration only and not by way of limitation. Several versions of this system have been described (Chien et al. 1991 PNAS USA 88:9578-9582; Yamada, M. et al. 2001 J Biochem (Tokyo) 130:157-65), and it is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to a Shp2 nucleotide sequence encoding Shp2, a Shp2 polypeptide, peptide or fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene, such as, for example, HBS or lacZ whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, Shp2 may be used as the bait gene product. Total genomic or cDNA sequences are fused to the DNA encoding an activation domain. This library and a plasmid encoding a hybrid of a bait Shp2 gene product fused to the DNA-binding domain are cotransformed into a yeast reporter strain, and the resulting transformants are screened for those that express the reporter gene. For example, and not by way of limitation, a bait Shp2 gene sequence, such as the open reading frame of Shp2 (or a domain of Shp2), can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait Shp2 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait Shp2 gene-GAL4 fusion plasmid into a yeast strain which contains a lacZ gene driven by a promoter which contains GAL4 activation sequence. A cDNA encoded protein, fused to GAL4 transcriptional activation domain, that interacts with bait Shp2 gene product will reconstitute an active GAL4 protein and thereby drive expression of the HIS3 gene. Colonies which express HIS3 can be detected by their growth on Petri dishes containing semi-solid agar based media lacking histidine. The cDNA can then be purified from these strains, and used to produce and isolate the bait Shp2 gene-interacting protein using techniques routinely practiced in the art.

### Assays for Compounds that Interfere with Shp2/Intracellular or Shp2/Transmembrane Macromolecule Interaction.

The macromolecules that interact with the Shp2 are referred to, for purposes of this discussion, as “binding partners”. These binding partners are likely to be involved in the Shp2 signal transduction pathway, and therefore, in the role of Shp2 in modulation of Shp2 interaction with leptin receptor. Therefore, it is desirable to identify compounds that interfere with or disrupt the interaction of such binding partners with Shp2 which may be useful in regulating the activity of the Shp2 and control Shp2 interaction with leptin receptor associated with Shp2 activity.

The basic principle of the assay systems used to identify compounds that interfere with the interaction between the Shp2 and its binding partner or partners involves preparing a reaction mixture containing Shp2 protein, polypeptide, peptide or fusion protein as described above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture. Alternatively, the test compound may be added at a time subsequent to the addition of the Shp2 moiety and its binding partner. Control reaction mixtures are incubated without the test compound or with a placebo. The formation of any complexes between the Shp2 moiety and the binding partner is then detected. The formation of a complex in the control reaction, but not in the reaction mixture containing the test compound, indicates that the compound interferes with the interaction of the Shp2 and the interactive binding partner. Additionally, complex formation within reaction mixtures containing the test compound and normal Shp2 protein may also be compared to complex formation within reaction mixtures containing the test compound and a mutant Shp2. This comparison may be important in those cases wherein it is desirable to identify compounds that disrupt interactions of mutant but not normal Shp2.

The assay for compounds that interfere with the interaction of the Shp2 and binding partners can be conducted in a heterogeneous or homogeneous format. Heterogeneous assays involve anchoring either the Shp2 moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance. In one embodiment, the test substance is added to the reaction mixture prior to the Shp2 moiety and interactive binding partner. In another embodiment, the test substance is added to the reaction mixture simultaneously with the Shp2 moiety and interactive binding partner. Alternatively, test compounds that disrupt preformed complexes, can be tested by adding the test compound to the reaction mixture after complexes have been formed. The various formats are described briefly below.

In a heterogeneous assay system, either the Shp2 moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the Shp2 gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed, for example, by washing and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface. In one embodiment, a labeled antibody specific for the initially non-immobilized species may be used. The antibody, in turn, may be directly labeled or indirectly labeled with a labeled anti-Ig antibody. Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected. Using an immobilized antibody specific for one of the binding components to anchor any complexes formed in solution, and a labeled antibody specific for the other partner to detect anchored complexes is contemplated. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the Shp2 moiety and the interactive binding partner is prepared in which either the Shp2 or its binding partners is labeled, but the signal generated by the label is quenched due to formation of the complex (see, for example, U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt Shp2/intracellular binding partner interaction can be identified.

In a particular embodiment, a Shp2 fusion can be prepared for immobilization. For example, the Shp2 or a peptide fragment, for example, corresponding to the Shp2 active site, can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with a radioactive isotope, for example ¹²⁵I, by methods routinely practiced in the art. In a heterogeneous assay, the GST-Shp2 fusion protein may be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the Shp2 gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-Shp2 fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the Shp2/binding partner interaction can be detected by adding the labeled antibody and measuring the radioactivity associated with the beads.

In another embodiment of the invention, these same techniques can be employed using peptide fragments that correspond to the binding domains of the Shp2 and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, a Shp2 gene product can be anchored to a solid material as described, above, by making a GST-Shp2 fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-Shp2 fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

### Cell- and Membrane-Based Screening Assays for Shp2 Modulators

Compounds, including but not limited to binding compounds identified via assay techniques such as those described in the preceding sections above can be tested for the ability to modulate Shp2 interaction with the leptin receptor. The assays described above can identify compounds which affect Shp2 activity. Compounds that bind to Shp2, inhibit binding of the natural ligand, and either activate signal transduction (agonists) or block activation (antagonists) are within the scope of the present invention. Compounds that bind to a natural ligand of Shp2 and neutralize ligand activity are also within the scope of the present invention. Compounds that affect Shp2 gene activity are also contemplated. Such compounds may be proteins or small organic molecules. However, it should be noted that the assays described can also identify compounds that modulate Shp2 signal transduction such as upstream or downstream signaling events. The identification and use of such compounds which affect another step in the Shp2 signal transduction pathway in which the Shp2 gene product is involved and, by affecting this same pathway may modulate the effect of Shp2 on the modulation of Shp2 interaction with leptin receptor are within the scope of the invention. Such compounds can be used as part of a method for the modulation of Shp2 interaction with leptin receptor.

Cell-based systems, membrane vesicle-based systems, and membrane fraction-based systems can be used to identify compounds which may act to modulate Shp2 interaction with leptin receptor. Such systems can include, for example, recombinant or non-recombinant cells, such as cell lines, which express the Shp2 gene. In addition, expression host cells genetically engineered to express a functional leptin receptor and to respond to activation by a natural Shp2 ligand can be used as an end point in the assay. Such activation can be measured by a chemical or phenotypic change, induction of another host cell gene, change in ion flux, phosphorylation of host cell proteins, etc.

In utilizing such cell-based systems, cells may be exposed to a compound suspected of exhibiting an ability to modulate Shp2 interaction with leptin receptor, at a sufficient concentration and for a time sufficient to elicit chemical or phenotypic change, induction of another host cell gene, change in ion flux, phosphorylation of host cell proteins, etc. in the exposed cells. After exposure, the cells can be assayed to measure alterations in the expression of the Shp2 gene. For example, cell lysates may be assayed for Shp2 mRNA transcripts or for Shp2 protein expressed in the cell. Compounds which regulate or modulate expression of the Shp2 gene are good candidates as modulators of Shp2 interaction with leptin receptor. Still further, the expression and/or activity of components of the signal transduction pathway of which Shp2 is a part, or the activity of the Shp2 signal transduction pathway itself can be assayed.

For example, after exposure, the cell lysates can be assayed for the presence of phosphorylation of host cell proteins, as compared to lysates derived from unexposed control cells. The ability of a test compound to inhibit phosphorylation of host cell proteins in these assay systems indicates that the test compound inhibits signal transduction initiated by Shp2 activation. The cell lysates can be readily assayed using a Western blot format well known in the art (see, for example, Glenney et al. 1988 J Immunol Methods 109:277-285; Frackelton et al. 1983 Mol Cell Biol 3:1343-1352). Alternatively, an ELISA format could be used in which a particular host cell protein involved in the Shp2 signal transduction pathway is immobilized using an anchoring antibody specific for the target host cell protein, and the presence or absence of a phosphorylated peptide residue on the immobilized host cell protein is detected using a labeled antibody (see, King et al. 1993 Life Sciences 53:1465-1472). In yet another approach, ion flux, such as calcium, potassium, sodium, bicarbonate, chloride ion flux, can be measured as an end point for Shp2 stimulated signal transduction.

In general, other cell-based screening procedures of the invention involve providing appropriate cells which express a Shp2 polypeptide. Such cells include cells from mammals, yeast, Drosophila or E. coli. In particular, a polynucleotide encoding the Shp2 is employed to transfect cells to thereby express a Shp2. The expressed Shp2 is then contacted with a test compound to observe binding, stimulation or inhibition of a functional response. One such screening procedure involves the use of melanophores which are transfected to express a Shp2 polypeptide. Such a screening technique is described in PCT WO 92/01810, published Feb. 6, 1992. Such an assay may be employed to screen for a compound which inhibits activation of Shp2 by contacting the melanophore cells which encode the Shp2 polypeptide with both a Shp2 ligand, and a compound to be screened. Inhibition of the signal generated by the ligand indicates that a compound is a potential antagonist for the Shp2, as it inhibits activation of the Shp2 polypeptide.

The technique may also be employed for screening of compounds which activate the Shp2 by contacting such cells with compounds to be screened and determining whether such compound generates a signal, as it activates the Shp2 polypeptide.

Other screening techniques include the use of cells which express a Shp2 in a system which measures extracellular pH changes caused by Shp2 activation. In this technique, compounds may be contacted with cells expressing a Shp2 polypeptide. A second messenger response, for example, signal transduction or pH changes, is then measured to determine whether the potential compound activates or inhibits the Shp2 polypeptide.

Another method involves screening for compounds which are antagonists, and thus inhibit activation of a Shp2 polypeptide by determining inhibition of binding of a labeled Shp2 ligand, in the cells which express Shp2. Such a method involves transfecting a eukaryotic cell with a DNA encoding a Shp2 polypeptide such that the cell expresses the Shp2 polypeptide. Alternatively a eukaryotic cell that expresses the Shp2 may be used. The cell is then contacted with a potential antagonist in the presence of a labeled form of a Shp2 ligand. The amount of labeled ligand bound to the Shp2 is measured. If the compound binds to the Shp2, the binding of labeled ligand to the Shp2 is inhibited as determined by a reduction of labeled ligand which binds to the Shp2. This method is called a binding assay.

Another such screening procedure involves the use of eukaryotic cells which are transfected to express Shp2 (or use of eukaryotic cells that express the Shp2). The cells are loaded with an indicator dye that produces a fluorescent signal when bound to calcium, and the cells are contacted with a test substance and a Shp2 agonist. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist (or agonist) for the Shp2 polypeptide.

Another such screening procedure involves use of eukaryotic cells which are transfected to express the Shp2 (or use of eukaryotic cells that express the Shp2), and which are also transfected with a reporter gene construct that is coupled to activation of the Shp2 polypeptide behind an appropriate promoter. Such reporter gene may be for example, luciferase or beta-galactosidase. The cells are contacted with a test substance and a Shp2 agonist and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the Shp2 polypeptide.

Another such screening technique for antagonists or agonists involves introducing RNA encoding a Shp2 polypeptide into Xenopus oocytes to transiently or stably express the Shp2 polypeptide. The oocytes are then contacted with a Shp2 ligand and a compound to be screened. Inhibition or activation of the Shp2 is then determined by detection of a signal, such as, cAMP, calcium, proton, or other ions.

Another rnethod involves screening for Shp2 polypeptide inhibitors by determining inhibition or stimulation of Shp2 polypeptide-mediated cAMP and/or adenylate cyclase accumulation or diminution. Such a method involves transiently or stably transfecting an eukaryotic cell with a Shp2 polynucleotide to express the Shp2 or using a eukaryotic cell that expresses the Shp2. The cell is then exposed to potential antagonists in the presence of Shp2 polypeptide ligand. The amount of cAMP accumulation is then measured, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist binds the Shp2 polypeptide, and thus inhibits Shp2 polypeptide activity, the levels of Shp2 polypeptide-mediated cAMP, or adenylate cyclase activity, will be reduced or increased.

The present invention also provides a method for determining whether a ligand not known to be capable of binding to Shp2 polypeptide can bind to such phosphatase. Such method comprises contacting a eukaryotic cell which expresses a Shp2 polypeptide with the ligand, under conditions permitting binding of candidate ligands to Shp2, and detecting the presence of a candidate ligand bound to the Shp2. The systems hereinabove described for determining agonists and/or antagonists may also be employed for determining ligands which bind to the Shp2.

Most pre-adipocyte cells (cultured cells) and human, primate and rodent primary adipocytes are capable of differentiating into mature adipocytes after induction by hormones and pharmaceutical agents. These hormones and agents may include (but are not limited to) insulin, dexamethasone, 3-isobutyl-1-methyl-xanthine (IBMX), long chain fatty acids, thiazolidinediones, prostaglandins, leukotrienes, eicosanoids, retinoids, RXRα agonists and any suitable combinations of all of the above (Kohanski et al. 1986 J Biol Chem 261:12272-12281; Brun et al. 1996 Genes Dev 10:974-984). The selected Shp2 regulators (agonists) are further investigated for their ability to mediate pre-adipocyte differentiation into adipocytes as measured by: (1) triglyceride accumulation, and/or (2) the expression of various marker genes such as aP2, adipsin, lipoprotein lipase or fatty acid synthase.

Candidate Shp2 agonist compounds identified through one or more of the in vitro screening assays described above are then administered to well known animal models such as, but not limited to, genetically or diet-induced obese mice (ob/ob, db/db, KkAy, agouti, high fat diet induced obese C57B1/6 or others), rats (fa/fa, ZDF, or others), hamsters (high fat diet induced obese Golden Syrian or other suitable strains) or monkeys (high fat diet induced obese cynamologous or African Green monkey) (see York “Genetic models of obesity” and Sclafani “Dietary models of obesity”, both in Obesity, Bjorntorp and Brodoff eds. J. B. Lippincott Company, 1992; McIntosh and Pederson, McNeill. eds. CRC press LLC, 337-398, 1999). Alternatively, these animals may also be used as primary screening tools. Compounds are administered in a pharmacologically acceptable vehicle to animals by intravenous, subcutaneous or intraportal injection, orally, or mixed with food or water, acutely or over an extended period of time. During the course of the study, various parameters such as water and food consumption, body weight gain and body temperature, are measured. Through tail vein bleeding blood is collected and plasma analyzed for glucose, insulin, free fatty acids, triglycerides and cholesterol. The animals are also tested for glucose tolerance and insulin sensitivity. The treated animals may also be scanned as compared to untreated obese animals for improvement in osteoarthritis of the joints. Compounds that act to reduce body weight or decrease plasma glucose and lipid levels or show increased glucose tolerance and insulin sensitivity and/or improvement in osteoarthitis of the joints are then selected for further study.

The invention described herein also includes pharmaceutically acceptable compositions of a Shp2 agonist for synthesis, storage, and delivery to a mammal (including humans) for the treatment of obesity and insulin resistance.

Many assays known to those skilled in the art of molecular biology, biochemistry, genetics, pharmacology and in vivo physiology can be used to screen and discover compounds that regulate Shp2 activity, regulate pre-adipocyte differentiation and prevent or ameliorate obesity, leptin resistance, and dys-metabolic syndrome.

### Other Compounds for Treating or Preventing Obesity.

For example, compounds for the treatment or prevention of a higher than desired total body weight or a higher than desired percentage of body fat may be identified from large libraries of both natural product or synthetic (or semisynthetic) extracts or chemical libraries according to methods known in the art.

Compounds of unknown or known function can be tested for their ability to increase Shp2 activity. For example, known compounds that are currently used to treat other conditions can be assayed to determine whether they increase Shp2 activity and thus are also useful for the treatment or prevention of obesity. Those skilled in the field or drug discovery and development will understand that the precise source of test extracts or compounds is not critical to the methods of the invention.

Accordingly, virtually any number of chemical extracts or compounds can be screened for their effect on reducing total body weight or body fat.

Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods.

Furthermore, if desired, any library or compound is readily modified using standard chemical, physical, or biochemical methods.

When a crude extract is found to inhibit angiogenesis and/or adipogenesis, further fractionation of the positive lead extract is necessary to isolate chemical constituent responsible for the observed effect. Thus, the goal of the extraction, fractionation, and purification process is the careful characterization and identification of a chemical entity within the crude extract.

Methods of fractionation and purification of such heterogeneous extracts are known in the art. If desired, compounds shown to be useful agents for the treatment or prevention of a higher than desired total body weight or a higher than desired percentage of body fat are chemically modified according to methods known in the art. Compounds identified as being of therapeutic value are subsequently analyzed using any standard animal model of angiogenesis, adipogenesis, or obesity known in the art.

### Other Assays and Animal Models for Testing Compounds of the Invention.

As described above, one or more candidate compounds can be tested for their effect on angiogenesis, adipogenesis, or obesity using the mouse model described herein. Alternatively, various genetically engineered obese mice can be used to determine the effect of compounds on obesity. Exemplary mice models of obesity include mice with a heterozygous or homozygous mutation in one or more of the following genes: obese (6b), diabetes (A), tubby (tub), fat, or Agouti, (see, for example, North, Current Opinion in Genetics & Development 9:283-288, 1999). A compound or a combination of compounds can also be tested in standard human clinical trials.

The efficacy of a compound in reducing excess body fat in animal or primate models or in humans can be monitored using standard methods. For example, the body mass index can be used to monitor a subject's weight. The amount of excess body fat can also be approximated by measuring subcutaneous fat (e.g., by measuring the thickness of a skin fold). If desired, a CAT scan or MRI can be used to more accurately measure the amount of body fat. Serum leptin levels should be proportional to the amount of body fat; thus, leptin levels can also be measured to monitor changes in body fat over time.

### In Vivo Obese Animal Model

In another preferred embodiment of the present invention C57B1/6 mice are fed a diet rich in fat (40%) and sucrose (40%) (see, York “Genetic models of obesity” and Sclafani “Dietary models of obesity”, both in Obesity, Bjomtorp and Brodoff eds. J B Lippincott Company, 1992; McIntosh and Pederson; McNeill. eds. CRC press LLC, 337-398, 1999; Farrelly et al. 1999 PNAS USA 96:14511-14516). Under these dietary conditions, C57B1/6 mice gain considerable body weight and become obese. These mice may be treated with Shp2 agonists (dose 1 to 100 mg/kg/day), administered in a pharmacologically acceptable vehicle (e.g. but not limited to 5% CM-cellulose) through intravenous, subcutaneous or intraportal injection, orally, or mixed with food or water, acutely or over an extended period of time. During the course of the study, various parameters such as water and food consumption, body weight gain, body temperature is measured by standard methods. Through tail vein bleeding, blood is collected in heparin-EDTA coated tubes to prevent clotting and blood plasma was separated and analyzed for glucose, free fatty acids, triglycerides and cholesterol using reagent kits available from Roche Diagnostics in a COBAS-MIRA instrument. Insulin and leptin are measured by commercially available ELISA kits. The animals are also tested for glucose tolerance and insulin sensitivity. This is performed by injecting a pre-determined dose of insulin (0.5 Units/kg in saline) or glucose (1 gm/kg in saline) and changes in glucose levels are monitored by tail vein bleed every 30 minutes. The compounds that lead to decreased levels of glucose after insulin injection and after a glucose load are considered insulin-sensitizing glucose lowering agents. Compounds that act to reduce body weight and or decrease glucose, lipid, or show increased glucose tolerance and insulin sensitivity are selected. The treated animals may also be scanned using suitable instruments for improvement in osteoarthritis of the joints.

Test compounds that prevent or ameliorate obesity, insulin resistance, are also tested in the disease models described above, in combination with an anti diabetic agent such as but not limited to metformin and sulfonylurea and/or a lipid lowering agent such as PPARα agonists (such as, but not limited to fenofibrate and gemfibrozil) and/or HMG CoA reductase inhibitors (such as, but not limited to pravastatin, lovastatin, simvastatin and atorvastatin). During the course of the study various parameters such as water and food consumption, body weight gain, body temperature and plasma glucose, insulin, free fatty acids, triglycerides and cholesterol levels are measured. The animals are also tested for glucose tolerance and insulin sensitivity. Compounds that act to reduce body weight and or decrease glucose, lipid, or show increased glucose tolerance and insulin sensitivity are selected for further characterization.

### Dosage and Formulation

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. In addition, materials, methods, and examples are illustrative only and are not intended to be limiting.

As used herein, the phrase “therapeutically effective” is intended to include an amount of a compound, or an amount of a combination of compounds, claimed effective to increase Shp2 activity and/or treat obesity, insulin resistance and/or hyperlipidemia.

As used herein, the term “prodrug(s)” is intended to include any covalently bonded carriers which release an active parent drug of the present invention in vivo when such a prodrug is administered to a mammalian subject. Prodrugs of the present invention are prepared by modifying functional groups present in the compound in such a way that the modifications are cleaved, either in routine manipulation or in vivo, resulting in the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate and benzoate derivatives of alcohol and amine functional groups in the compounds of the present invention.

As used herein, the phrase “pharmaceutically acceptable” is employed to refer to those compounds, materials, compositions and/or dosage forms which are, within the scope of sound medical judgment, suitable for use in contact with the tissues of mammals, including human beings, without excessive toxicity, irritation, allergic response, or other problem or complication commensurate with a reasonable benefit/risk ratio.

As used herein, the phrase “anti-diabetic agent” refers to a compound that will improve insulin resistance and decrease plasma glucose levels in patients with diabetes. Representative compounds within the scope of the present invention include but are not limited to metformin, rosiglitazone, and pioglitazone.

As used herein, the phrase “lipid-lowering agent” refers to a compound that will lower plasma lipid levels—cholesterol and triglycerides, in patients suffering from hyperlipidemia and/or cardiovascular disease. Representative compounds within the scope of the present invention include but are not limited to pravastatin, simvastatin, atorvastatin, and gemfibrozil.

As used herein, the phrase “administered in combination”, and the terms “combination” or “combined” when referring to compounds, components, or compositions described herein, means the compounds, components, or compositions are administered concurrently to the mammal being treated. When administered in combination each compound, component, or composition may be administered in any order at the same time or sequentially in any order or at different points in time, so as to provide the desired therapeutic effect.

As used herein the terms “modulate or modulates” refer to an increase or decrease in the amount, quality or effect of a particular activity or protein.

A therapy of the invention may be administered to humans, domestic pets, livestock, or other animals with a pharmaceutically acceptable diluent, carrier, or excipient, in unit dosage form.

The compounds optionally may be administered as pharmaceutically acceptable salts, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

The chemical compounds for use in such therapies may be produced and isolated as described herein or by any standard technique known to those in the field of medicinal chemistry. Conventional pharmaceutical practice may be employed to provide suitable formulations or compositions to administer the identified compound to patients suffering from a higher than desired total body weight or a higher than desired percentage of body fat. Administration may begin before or after the patient is symptomatic.

Any appropriate route of administration may be employed. Preferably, the therapy is administered using a controlled-release microchip, microparticle extended-release formulation, polymeric nanoparticle, or transdermal delivery system (as described, for example, in LaVan et al., Nature Reviews 1:77-84, 2000 or Santini et al., Nature 397:335-338, 1999). Administration of the compounds may also be oral, topical parenteral, intravenous, intraarterial, subcutaneous, intramuscular, intracranial, intraorbital, ophthalmic, intraventricular, intracapsular, intraspinal, intracisternal, intraperitoneal, or intranasal. Alternatively, the compounds may be administered as part of a suppository. Preferably, the chemical compounds for use in treatment of body weight disorders are capable of traversing the blood-brain barrier. Therapeutic formulations may be in the form of liquid solutions or suspensions; for oral administration, formulations may be in the form of tablets or capsules; and for intranasal formulations, in the form of powders, nasal drops, or aerosols. The compounds in a combination therapy may be administered simultaneously or sequentially. For example, one or more compounds in a combination therapy can be administered until the compound(s) normalize the blood vessel network of fat tissue and thereby increase the accessibility of the fat tissue to other therapeutic agents, and then one or more additional compounds can be administered instead of, or in addition to, the originally administered compound(s). The dosage of the therapeutic compounds in a pharmaceutically acceptable formulation depends on a number of factors, including the size and health of the individual patient. The dosage to deliver may be determined by one skilled in the art. For example, compounds that are administered as part of a combination therapy of the invention are typically administered at a dose equal to or at least 25, 50, or 75% lower than the corresponding dose for the compound when it is used individually.

Methods well known in the art for making formulations are found, for example, in “Remington: The Science and Practice of Pharmacv”, 19^{th} ed. ed. A. R. Gennaro AR., 1995, Mack Publishing Company, Easton, Pa.).

Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene lauryl ether, glycocholate and deoxycholate, or may be oily solutions for administration in the form of nasal drops, or as a gel.

If desired, treatment with a compound identified according to the methods described above, may be combined with more traditional therapies for decreasing total body weight or percentage of body fat (e.g., diet, exercise, or appetite suppressant).

A suitable Shp2 agonist compound can be administered to patients to treat obesity and other metabolic disorders as the compound alone and/or mixed with an acceptable carrier in the form of pharmaceutical formulations. Those skilled in the art of obesity, insulin resistance, leptin resistance and hyperlipidemia can easily determine the dosage and route of administration of the compound to mammals, including humans, in need of such treatment. The route of administration may include but is not limited to oral, rectal, transdermal, buccal, transnasal, subcutaneous, intramuscular, intradermal, intravenous, or intestinal administration. The compound is formulated according to the route of administration based on acceptable pharmacy practice (Fingl et al. 1975 in The Pharmacological Basis of Therapeutics Ch. 1, p. 1; Remington's Pharmaceutical Sciences, 18th ed., Mack Publishing Co, Easton, Pa., 1990).

In combination therapy, the dose and route of administration of the second or third drug (anti-diabetic or lipid lowering drugs) will depend on the drug chosen and the severity of insulin resistance, type 2 diabetes and/or hyperlipidemia.

Pharmaceutically acceptable Shp2 agonist compositions can be administered in oral dosage forms such as tablets, capsules (each of which includes sustained release or timed release formulations), pills, powders, granules, elixirs, tinctures, suspensions, syrups, and emulsions. The compositions can also be administered in intravenous (bolus or infusion), intraperitoneal, subcutaneous, or intramuscular form, all using dosage forms well known to those of ordinary skill in the pharmaceutical arts. A composition may be administered alone, but generally will be administered with a pharmaceutical carrier selected on the basis of the chosen route of administration and standard pharmaceutical practice.

The dosage regimen for the composition of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disease state.

By way of general guidance, the daily oral dosage of the active ingredient, when used for the indicated effects, will range between about 0.001 to 1000 mg/kg of body weight, preferably between about 0.01 to 100 mg/kg of body weight per day, and most preferably between about 1.0 to 20 mg/kg/day. Intravenously, the most preferred doses will range from about 1 to about 10 mg/kg/minute during a constant rate infusion. The composition of this invention may be administered in a single daily dose, or the total daily dosage may be administered in divided doses of two, three, or four times daily.

The composition of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The composition is typically administered in a mixture with suitable pharmaceutical diluents, excipients, or carriers (collectively referred to herein as pharmaceutical carriers) suitably selected with respect to the intended form of administration, that is, oral tablets, capsules, elixirs, and syrups, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, and sorbitol; for oral administration in liquid form, the oral drug components can be combined with any oral, non-toxic, pharmaceutically acceptable inert carrier such as ethanol, glycerol, and water. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, and waxes. Lubricants used in these dosage forrns include sodium oleate, sodium stearate, magnesium stearate, sodium benzoate, sodium acetate, and sodium chloride. Disintegrators include, but are not limited to, starch, methyl cellulose, agar, bentonite, and xanthan gum.

The composition of the present invention may also be administered in the form of liposome delivery systems, such as small unilamellar vesicles, large unilamellar vesicles, and multilamellar vesicles. Liposornes can be formed from a variety of phospholipids, such as cholesterol, stearylamine, or phosphatidylcholines.

Since prodrugs are known to enhance numerous desirable qualities of pharmaceuticals (i.e., solubility, bioavailability, manufacturing, etc.) the compounds of the present invention may be delivered in prodrug form. Thus, the present invention is intended to cover prodrugs of the presently claimed compounds, methods of delivering the same and compositions containing the same.

The compositions of the present invention may also be coupled with soluble polymers as targetable drug carriers. Such polymers can include polyvinyl-pyrrolidone, pyran copolymer, polyhydroxypropyl-methacrylamide-phenol, polyhydroxyethylaspartamidephenol, or polyethyleneoxide-polylysine substituted with palmitoyl residues. Furthermore, the composition of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The compositions of the present invention may be conjugated with a Blood-Brain Barrier delivery targeting vector such as, for example, avidin-biotin linked chimeric peptide, monoclonal antibody to the transferrin receptor, transferrin, L-Glutamate; short natural-derived peptides that are able to cross efficiently the BBB without compromising its integrity; antibody-avidin fusion protein, etc. (Song B. et al. 2002 J Pharmacol Exp Ther 301:605-10; Pardridge W. M. et al. 2001 Jpn J Pharmacol 87:97-103 and references therein; Liao G. S. et al. 2001 J Nat Toxins 10:291-7; Sakaeda T. et al. 2001 J Drug Target 9:23-37; Rousselle C. et al. 2001 J Pharmacol Exp Ther 296:124-31; Penichet M. L. et al. 1999 J Immunol 163:4421-6; or lipid nanoparticles as described, for example in Olbrich C. et al. 2002 J Drug Target 10:387-96.

Dosage forms (pharmaceutical compositions) suitable for administration may contain from about 1 milligram to about 100 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivative, magnesium stearate, and stearic acid. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration cain contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solution for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington: The Science and Practice of Pharmacy, Nineteenth Edition, Mack Publishing Company, 1995, a standard reference text in this field

Representative useful pharmaceutical dosage forms for administration of the compound of this invention can be illustrated as follows:

Capsules: A large number of unit capsules can be prepared by filling standard two-piece hard gelatin capsules with 100 milligrams of powdered active ingredient, 150 milligrams of lactose, 50 milligrams of cellulose, and 6 milligrams magnesium stearate;

Soft Gelatin Capsules: A mixture of active ingredient in a digestable oil such as soybean oil, cottonseed oil or olive oil may be prepared and injected by means of a positive displacement pump into gelatin to form soft gelatin capsules containing 100 milligrams of the active ingredient. The capsules should be washed and dried.

Tablets: Tablets may be prepared by conventional procedures so that the dosage unit, for example is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

Injectable: A parenteral composition suitable for administration by injection may be prepared by stirring 1.5% by weight of active ingredient in 10% by volume propylene glycol and water. The solution should be made isotonic with sodium chloride and sterilized.

Suspension: An aqueous suspension can be prepared for oral administration so that, for example, each 5 mL contains 100 mg of finely divided active ingredient, 20 mg of sodium carboxymethyl cellulose, 5 mg of sodium benzoate, 1.0 g of sorbitol solution, U.S.P., and 0.025 mL of vanillin or other palatable flavoring.

### EXAMPLE 1

The following example relates to the discovery that specific ablation of the Shp2 gene in forebrain neurons caused resistance to leptin in mice. The leptin resistance was characterized by early-onset obesity and increased serum levels of leptin, insulin and triglycerides. The mutant animals, however, did not show hyperphagia and were hyperglycemic in the fed state while hypoglycemic in the fasting state. Furthermore, the mutant mice developed hepatomegaly, with increased lipid content, up-regulated anabolic gene expression and impaired catabolic gene expression in the liver. Basal and leptin-induced Stat3 activation in the hypothalamus was enhanced in the absence of Shp2. Thus, although Shp2 has a minor negative role in modulating Stat3 activation by leptin, the primary function of Shp2 in the hypothalamus appears to be in promoting the metabolic activity of leptin, independent of its anorectic effect, in energy balance.

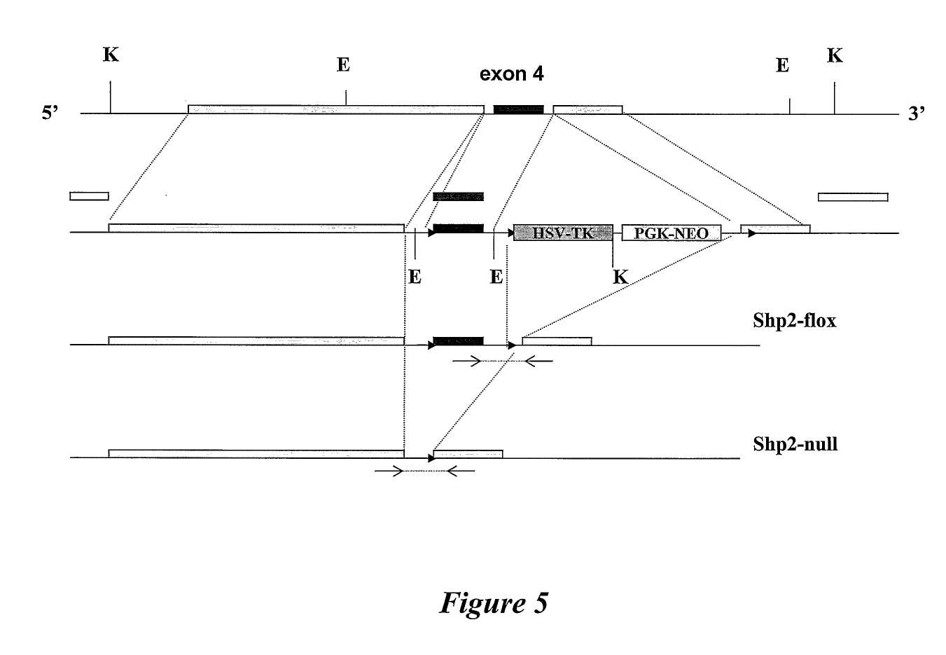

A conditional Shp2 mutant (Shp2^{flox}) allele was created by introducing two loxP sites into introns flanking exon 4 which codes for amino acid residues 111-176 of Shp2, using the Cre-loxP technology (FIG. 5). Deletion of exon 4 introduced a frame-shift mutation and created a premature stop codon immediately.

To generate a conditional Shp2^{flox} mutant allele, a targeting construct was engineered, with neomycin-resistance (neoR), thymidine kinase (TK) and diphtheria toxin (DT-A) genes as selective markers (FIG. 5). R1 embryonic stem (ES) cells were transfected with the linearized targeting construct by electroporation and selected in DMEM containing G418 for homologous recombination. PCR analysis was used for screening of ES cell clones. Southern blot analysis identified ES clones with homologous recombination at the left and right arms and excluded those with the unwanted central-arm recombination. A correctly targeted ES cell clone was transiently transfected with a Cre expression plasmid (pBS185) by electroporation and selected in DMEM medium supplemented with 1-(2-deoxy-2-fluoro-D-arabinofurnaosyl)-5-iodouracil (FIAU), to remove the neo-TK cassette. After confirmation by Southern blot analysis, three ES cell clones with a loxP-floxed Shp2 allele (Shp2^{flox}) were injected into blastocysts to generate chimeric mice. Male chimeras displaying almost 100% agouti color were bred with C57B1/6 females to produce F1 generations. Germ-line transmission of the Shp2^{flox/+} allele was achieved from all three ES cell clones injected, and backcrossed with C57B1/6 for at least 3 generations. Shp2^{flox/+} mice were subsequently bred for two generations with CamK2a-Cre transgenic mice (strain R1ag#5) in the C57B1/6 background (Dragatsis, I. & Zeitlin, S. 2000 Genesis 26:133-5; Rios M. et al. 2001 Mol Endocrinol 15:1748-57). In this study, Shp2^{flox/flox} mice were used as wild-type controls, Cre/+: Shp2^{flox/+} as heterozygous, and Cre/+:Shp2^{flox/flox} (CaSKO) mice as homozygous mutants.

To detect the Shp2^{flox} allele, a forward primer (5′-ACG TCA TGA TCC GCT GTC AG-3′) (SEQ ID NO: 1) in the exon 4 and a reverse primer (5′-ATG GGA GGG ACA GTG CAG TG-3′) (SEQ ID NO: 2) in the intron 4 were used. For the Shp2null allele, a forward primer (5′-CAG TTG CAA CTT TCT TAC CTC-3′) (SEQ ID NO: 3) in the intron 3 and a reverse primer (5′-GCA GGA GAC TGC AGC TCA GTG ATG-3′) (SEQ ID NO: 4) within intron 4 were used. For Southern blot analysis, both 5′ and 3′-external probes were used as shown. In addition, a probe for the central arm was used for detection of unwanted central-arm recombinants.

The brain-derived neurotrophic factor, BDNF primers used are 5′-CTG ACA CTTT TGA GCA CGT CAT C-3′ (SEQ ID NO: 5) and 5′-AGG CTC CAA AGG CAC TTG ACT-3′ (SEQ ID NO: 6), following a previously published design (Baker-Herman, T. L. et al. 2004 Nat Neurosci 7:48-55).

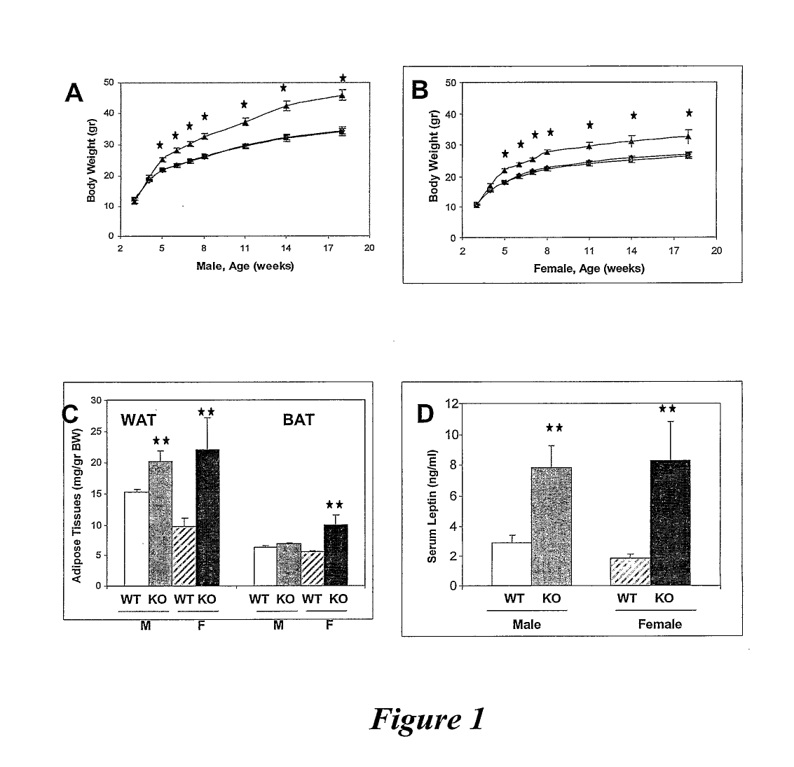

To investigate Shp2 function in the brain, a mouse model of brain-specific Shp2 knockout (CamK2a-Cre:Shp2^{flox/flox} or CaSKO) was created by crossing Shp2^{flox/flox} mice with CamK2a-Cre transgenic mice (I. Dragatsis, & S. Zeitlin, 2000 Genesis 26:133-5). Previous analysis demonstrated that in this transgenic line, Cre recombinase was expressed in postmitotic neuronal cells but not in glial cells after postnatal day 5 (P5) (M. Rios et al., 2001 Mol Endocrinol 15:1748-57). PCR analysis confirmed a Cre-mediated specific recombination of the Shp2^{flox} allele in neuronal cells in the forebrain such as cerebral cortex and hypothalamus but not in other tissues. Immunoblot analysis using a specific anti-Shp2 antibody demonstrated a decrease by 50-70% of total Shp2 protein level in the forebrain lysate of CaSKO mice at P21. Double immunohistochemical staining of Cre and NeuN confirmed the restricted expression of Cre to neuronal cells in cerebral cortex and hypothalamus (FIG. 1C).

For measurement of mouse body weights, food intake and anatomical analyses, all data were collected between 10:00 AM and 12:00 PM daily. For hormone studies, mouse sera were collected restrictively from 10:00 AM to 11:00 AM. Blood glucose was determined on whole venous blood using an automatic glucometer (One Touch Basic, Lifescan) The serum leptin/insulin measurements were conducted by ELISA (Crystal Chem Inc.), corticosterone analysis by EIA (R&D Systems), and TSH and GH by RIA (UCLA-Harbor Medical Center). Serum triglyceride was determined by the Diagnostic laboratory of UCSD Animal Care Program. Except for mice in breeding or behavior examination, all mice analyzed here were single-housed after weaning at day 21 after birth (P21). All animal procedures were approved by The Burnham Institutional Animal Care and Use Committee.

The most prominent and immediately noticeable phenotype of the CaSKO mice was an early-onset obesity and accelerated increase of body weight in both males and females, while heterozygous animals appeared normal (FIG. 1A and B). At 8 weeks of age, male mutants gained 28% more and females 21% more over their wild-type and heterozygous littermates (P<0.0001). Overall, both male and female CaSKO adult mice weighed 30-50% heavier than their age- and sex-matched littermates on regular chow food. The abnormal increase of body weight correlated with the development of early-onset obesity in CaSKO mice. At 8 weeks of age, male mutants had 32% more white adipose tissue (WAT) than controls, while females had 12.3% more (P<0.01, FIG. 1C). Similarly, females had 80% more brown adipose tissue (BAT, P<0.01) and males had 9% (P=0.184). Consistent with the increase of fat tissue, serum triglyceride levels were significantly higher in mutants, with nearly 25% more in both males and females (P<0.05 each, Table 1). Notably, the serum leptin levels in CaSKO mice were significantly increased by 2.7 fold in the males, 4.6 fold in the females, (P<0.01 each, FIG. 1D), suggesting a resistance to leptin.

For immuno-precipitation, immunoblotting, and immunohistochemistry studies, polyclonal antibodies against ObRb protein and against phosphorylated Tyr985 (pY985) of ObRb were from Alpha Diagnostic International, Inc. Others antibodies used were polyclonal antibodies against C-terminal tail of Shp2 (Santa Cruz), phospho-Erk1/2 or Erk1/2 kinases, phospho-tyrosyl (pY)-Stat3 or STAT3 (Cell Signaling), Cre recombinase (Novagen), and a monoclonal antibody against NeuN (Chemicon). The Secondary antibodies used in immuno- staining were AlexaFluor594 anti-rabbit IgG and AlexaFluor488 anti-mouse IgG (1:200 dilution, Molecular Probes). Reagents for Oil-Red-O and periodic acid-Schiff staining were from PolyScientific. Recombinant mouse leptin was from the National Hormone & Peptide Program. For Northern blot, liver RNA was extracted by Trizol reagent (Invitrogen). For real-time RT-PCR, hypothalamic RNA was extracted by RNeasy kits (Qiagen), and the reaction was performed by LightCycler machine (Roche) with a SYBR-Green RT-PCR kit (Qiagen). All results were presented as comparative analysis for littermates of wild-type and CaSKO mice.

ObRb was co-precipitated with Shp2 in hypothalamic lysates prepared from wild-type mice after leptin treatment for 15 min, indicating a direct involvement of Shp2 in leptin signaling proximal to the ObRb receptor in the hypothalamus. Both the basal and leptin-induced tyrosine phosphorylation levels of Stat3 were not reduced or even slightly enhanced in the hypothalamus of CaSKO mice compared to the controls, supporting the notion that Shp2 down-regulates the ObRb-Stat3 pathway. Nevertheless, the obesity and increased serum levels of leptin in CaSKO mice strongly argues for a positive role of Shp2 in leptin signaling. A strong support to this notion is the data showing that leptin-induced phospho-Erk signal in arcuate nucleus of hypothalamus was dramatically reduced in CaSKO mice compared to control, while nuclear translocation of Stat3 was not apparently impaired in the same area upon leptin administration. Thus, Shp2 acts to promote leptin signaling mainly through activation of the Erk pathway, leading to leptin resistance in CaSKO mice.

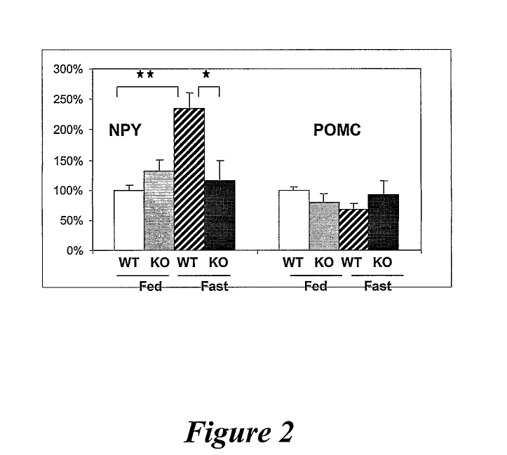

Mice at age of 8 weeks were either fed ad libitum, or fasted for 20 hrs before total RNA extraction from hypothalami. Real time RT-PCR was performed according to a previously published protocol (S. H. Bates et al. 2003 Nature 421:856-9). In measuring the hypothalamic signals downstream of leptin receptor, RT-PCR analysis demonstrated no significant changes in the mRNA levels of POMC between controls and CaSKO mice. However, the NPY mRNA level increased 2-3 fold in control mice after 20 hr fasting, with no increase detected in CaSKO mice (FIG. 2). This result indicates that Shp2 is required for leptin activation of NPY in a Stat3-independent fashion.

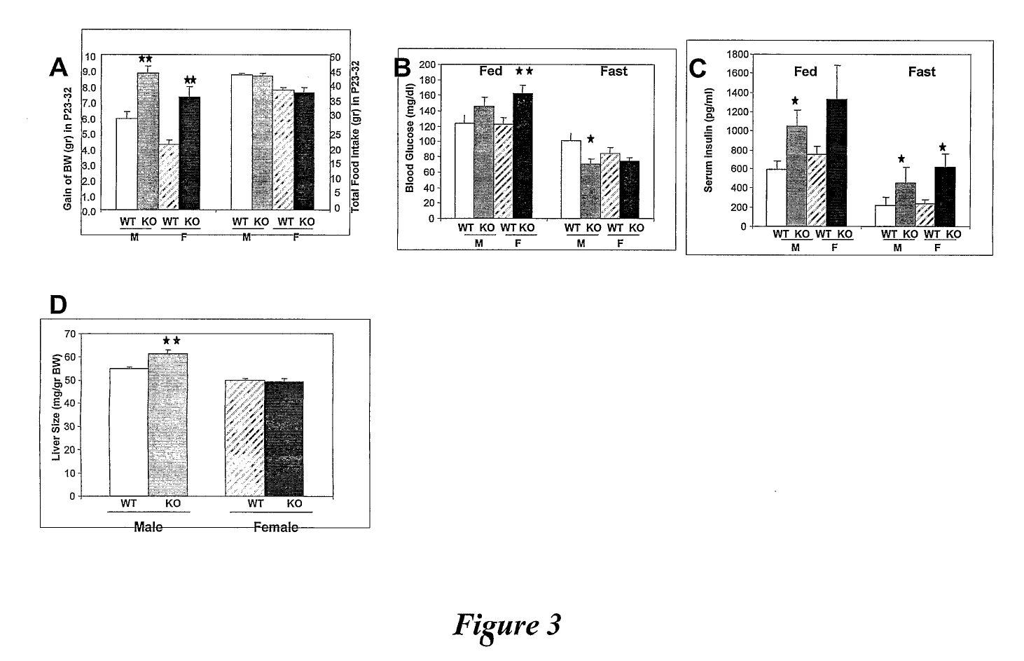

To explore the physiological mechanism of the obese phenotype, total food intake and body weight increase for the period of P23-32 was assessed, a time window around the onset of obesity (FIG. 3A). When evaluated at P23, the body weights of control and CaSKO mice were indistinguishable in both males and females. During the subsequent 10 days, mutant males gained 150% of body weight compared to controls and mutant females had a body weight increase at 172% of the controls (n≧8 each group, P<0.0002), despite the fact that food intake between controls and mutants were equal or even slightly less for the mutants (FIG. 3A). Therefore, CaSKO mice were not hyperphagic at the onset of obesity. We also compared food intake between controls and CaSKO mice at 8 weeks of age, after development of obesity, and found no significant difference (Table 1). Together, these data suggest that CaSKO mice did not develop hyperphagia and that the obesity was rather due to alteration of metabolism upon deletion of Shp2 in the brain. Consistently, the body temperature of CaSKO mice was significantly lower than the controls (35.8 versus 36.5° C., Table 1).

We then investigated glucose homeostasis and found that in fed state, CaSKO mice were hyperglycemic at 8 weeks of age (FIG. 3B). However, upon fasting for 20 hr, the CaSKO mice became hypoglycemic compared to littermate controls (FIG. 3B). In contrast, hyperinsulinemia was detected in mutant mice in both fed and fasting state (FIG. 3C). One plausible explanation is that the Shp2 mutation in CaSKO mice changed metabolic pathways in favor of anabolism over catabolism, resulting in more blood glucose loss under the fasting stress.

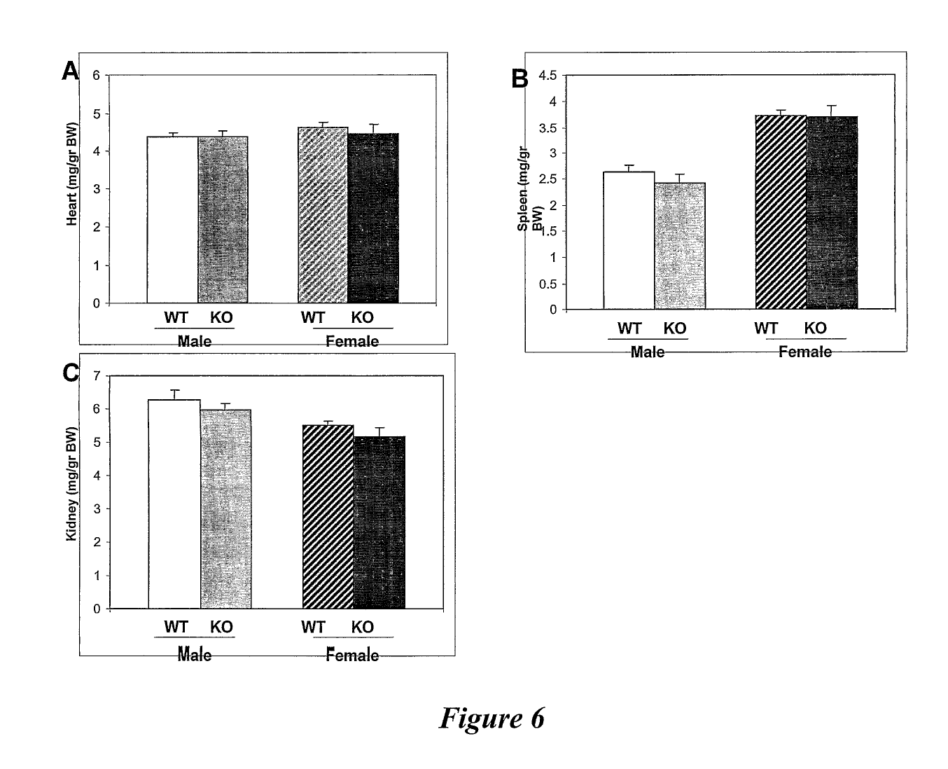

The size and weights of most organs, such as heart, kidney, and spleen, appeared normal (FIG. 6). Strikingly, the liver size in male mutants, however, was significantly increased by 12% (P=.002, FIG. 3D). Oil-Red-O staining on cryo-sections from 15-week-old CaSKO mice showed fatty liver. In addition to fat storage within hepatic cells, which results in fatty liver, triglycerides were secreted from hepatocytes but were abnormally accumulated in the liver, contributing to the phenotype of hepatomegaly. Indeed, Periodic Acid-Schiff (PAS) staining displayed elevation of glycogen deposition in the liver of CaSKO mice. Therefore, deletion of the Shp2 gene in neuronal cells in the forebrain resulted in a change in hepatic glucose/lipid metabolism.

To assess the expression of genes controlling lipid metabolism in the liver, total RNAs were extracted from mouse livers at the age of P28 (just before the onset of obesity). Northern blot analysis demonstrated that expression of lipogenic genes, such as stearoyl-CoA desaturase-1 (SCD-1)and fatty acid synthase (FAS), was up-regulated, while a lipolytic gene coding for very long chain acyl-CoA dehydrogenase (VLCAD) was down-regulated. As these changes were detected in young mice before the onset of obesity, it is reasonable to conclude that altered expression of the enzymes is the cause, rather than the consequence, of obesity in CaSKO mice.

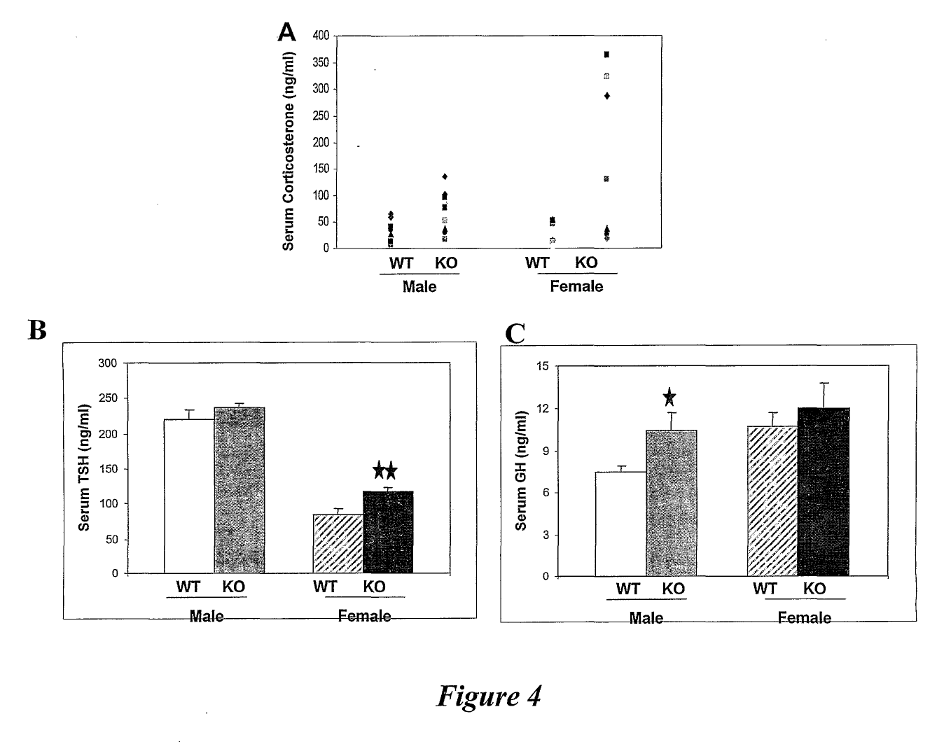

Leptin-initiated signals in the hypothalamus act through pituitary hormones in control of metabolism, and ob/ob mice display severe dysfunctions in the hypothalamus-pituitary axis (J. M. Friedman, & J. L. Halaas, 1998 Nature 395:763-7). CaSKO mice exhibited hypersecretion of glucocorticoids (FIG. 4A), with some mutants exhibiting the “moon face” due to redistribution of fat. In contrast to ob/ob mice that show impaired functions in the hypothalamus-pituitary-thyroid axis (Barsh, G. S. & Schwartz, M. W. 2002 Nat Rev Genet 3:589-600), CaSKO mice had higher serum levels, of thyroid stimulating hormone (TSH) (FIG. 4B), possibly due to enhanced Stat3 activation (Harris M. et al. 2001 J Clin Invest 107:111-20). Consistent with the elevated TSH levels, male homozygous mutants were more aggressive than the wild-type or heterozygous animals. CaSKO mice displayed increased linear growth, the snout-anus length compared to controls (Table 1) and, consistently, hypersecretion of growth hormone (GH) was observed in male mutants and to the lesser extent in females (FIG. 4C). In ob/ob mice, GH is hyposecreted and the snout-anus length is shorter than wild-type mice. Finally, CaSKO mice displayed severe impairment in reproduction (45% breeding efficiency, Table 1), while ob/ob mice were completely sterile.

TABLE 1
Phenotypic Characterization of CaSKO Mice

| | | | | P value |
|---|---|---|---|---|
| Genotype | F/F | F/+, Cre | F/F, Cre | (Ctl & KO) |
| Weight (male), g* | 27.1 + 0.5 | 26.9 + 0.6 | 34.5 + 1.4 | 0.0002 |
| Weight (female), g** | 23.2 + 0.5 | 22.8 + 0.5 | 27.9 + 1.3 | 0.001 |
| Feeding (g per animal)* | 5.0 + 0.2 | 4.9 + 0.4 | 5.7 + 1.4 | 0.184 |
| Serum Triglycerides (mg/dl)* | 146.0 + 9.3 | ND | 173.6 + 7.3 | 0.039 |
| serum Triglycerides (mg/dl)** | 81.9 + 3.6 | ND | 97.9 + 7.0 | 0.047 |
| Snout-anus length (mm)* | 91 + 1 | 90 + 1 | 98 + 1 | 0.002 |
| Body temperature*** | 36.5 + 0.2 | ND | 35.8 + 0.2 | 0.019 |
| Fertility (female)** | 10/10 (100%) | 20/20 | 8/12 (45%)Δ | NA |

*8-week-old male mice
**8-week-old females
***10-week-old males
Δfor a period of 3 months, 8 out of 12 female KO mice delivered pups, and the average litter number was 45% of controls.

In summary, we report here that deletion of Shp2 in the forebrain caused resistance to leptin activity in mice, leading to development of obesity and fatty liver in CaSKO mice. It is possible that chronic inhibition of Shp2 activity in the human brain might be a molecular basis for progression of obesity in aged obese subjects. Although mostly known for its anorectic effect, leptin stimulates a metabolic response that cannot be explained by its control of food intake alone, i.e. leptin administration into ob/ob mice and humans leads to reduction of lipid in liver and adipose tissues, and improves insulin sensitivity (Kamohara, S. et al. 1997 Nature 389:374-7; Levin, N. et al. 1996 PNAS USA 93:1726-30; J. L. Halaas et al., 1995 Science 269:543-6; Pelleymounter M. A. et al. 1995 Science 269:540-3; Shimomura, I. et al. 1999 Nature 401:73-6; Farooqi I. S. et al. 2002 J Clin Invest 110:1093-103). Shp2 was identified as a critical component in transducing the metabolic signal of leptin through control of SCD-1 expression in the liver. Deletion of brain-derived neurotrophic factor (BDNF) in the postnatal brain also caused obesity and energy imbalance (Rios M. et al. 2001 Mol Endocrinol 15:1748-57; Kernie, S. G. et al. 2000 EMBO J 19:1290-300; Xu B. et al. 2003 Nat Neurosci 6:736-42).

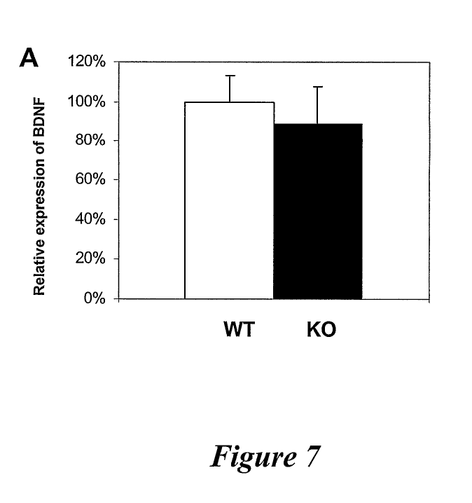

RNA was extracted from hypothalami of 8-week-old mice and analyzed by real time RT-PCR. Expression of hypoxanthine guanine phosphoribosyl transferase (HPRT) gene was used as an internal control. The result presented was from at least 3 pairs of littermates of wild-type and knockout mice, and the P value is greater than 0.5. The expression levels of BDNF mRNA in the hypothalamus was found no different between control and CaSKO mice (FIG. 7), arguing against a possibility that deletion of Shp2 in the brain may lead to development of resistance to BDNF. In comparison with db/db and ObRb-Tyr1138Ser knockin mice (Friedman, J. M. & Halaas, J. L. 1998 Nature 395:763-70; Bates S. H. et al. 2003 Nature 421:856-9), the phenotype of CaSKO mice described above manifested a typical disruption of leptin signaling. The striking difference in the phenotypes between CaSKO and NIRKO mice further supports that Shp2 has a primary function in signaling for leptin rather than insulin in the hypothalamus (Bruning J. C. et al. 2000 Science 289:2122-5). Thus, Shp2 plays a critical role in the brain control of body weight, glucose homeostasis and energy balance in postnatal mammals.

The present invention is not to be limited in scope by the specific embodiments described that are intended as single illustrations of individual aspects of the invention. Functionally equivalent methods and components in addition to those shown and described herein will become apparent to those skilled in the art from the foregoing description and accompanying drawings. Such modifications are intended to fall within the scope of the appended claims.

All publications, patent applications, and patents mentioned in this specification are herein incorporated by reference to the same extent as if each independent publication, patent, or patent application was specifically and individually indicated to be incorporated by reference.

1-6. (canceled)

7. A method of treating, stabilizing or preventing a lower than desired total body weight or a lower than desired percentage of body fat in a mammal comprising:
selecting a mammal in need of treatment for having a lower than desired total body weight or a lower than desired percentage of body fat; and
administering to the mammal a compound that decreases Shp2 activity.

8. The method of claim 7, wherein said compound decreases Shp2 activity in neurons of said mammal.

9. The method of claim 8, wherein said compound decreases Shp2 activity in neurons of forebrain of said mammal.

10. The method of claim 9, wherein said compound decreases Shp2 activity in neurons of hypothalamus of said mammal.

11. The method of claim 7, wherein said compound decreases a level of Shp2 mRNA or protein, an activity of Shp2, a half-life of Shp2 mRNA or protein, or a binding of Shp2 to a leptin receptor.

12. The method of claim 11, wherein said compound is a Shp2 antagonist.

13. (canceled)

14. A screening method for determining a compound useful for treating, stabilizing, or preventing a lower than desired total body weight or a lower than desired percentage of body fat in a mammal, said method comprising
contacting a cell with said compound; and
measuring Shp2 activity in said cell in the presence and absence of the compound, wherein the compound is determined to treat, stabilize, or prevent a lower than desired total body weight or a lower than desired percentage of body fat if the compound decreases the level of Shp2 activity.

15-25. (canceled)

26. A genetically modified mouse comprising a disrupted Shp2 gene, wherein said genetically modified mouse is homozygous for said disrupted Shp2 gene, and wherein said genetically modified mouse exhibits an increased body weight in comparison to a mouse that does not have a disrupted Shp2 gene.

27. The genetically modified mouse of claim 26, wherein said Shp2 gene is disrupted in the forebrain of said mouse.

28. The genetically modified mouse of claim 26, wherein said mouse has an early onset obesity.

29. The genetically modified mouse of claim 26, wherein said mouse has a resistance to leptin.

30. The genetically modified mouse of claim 26, wherein Shp2 protein level is decreased by 50-70% in the forebrain of said mouse.

31. The genetically modified mouse of claim 26, wherein triglyceride levels are increase in the serum of said mouse.

32. The genetically modified mouse of claim 26, wherein said Shp2 gene is absent in the forebrain of said mouse.

33. A method of screening compounds for preventing or ameliorating obesity, comprising:
(a) providing a genetically modified mouse comprising a disrupted Shp2 gene, wherein said genetically modified mouse is homozygous for said disrupted Shp2 gene, and wherein said genetically modified mouse exhibits accelerated increase of body weight;
(b) administering a test compound to said genetically modified mouse;
(c) determining the effect of said test compound on the body weight of said genetically modified mouse; and
(d) correlating a decrease in the body weight of said genetically modified mouse with an anti-obesity effect of said test compound.


SEQUENCE LISTING (6 sequences)

SEQ ID NO: 1 - DNA, 20 bp, Artificial Sequence
  synthetic primer
acgtcatgatccgctgtcag

SEQ ID NO: 2 - DNA, 20 bp, Artificial Sequence
  synthetic primer
atgggagggacagtgcagtg

SEQ ID NO: 3 - DNA, 21 bp, Artificial Sequence
  synthetic primer
cagttgcaactttcttacctc

SEQ ID NO: 4 - DNA, 24 bp, Artificial Sequence
  synthetic primer
gcaggagactgcagctcagtgatg

SEQ ID NO: 5 - DNA, 23 bp, Artificial Sequence
  synthetic primer
ctgacacttttgagcacgtcatc

SEQ ID NO: 6 - DNA, 21 bp, Artificial Sequence
  synthetic primer
aggctccaaaggcacttgact